(19) United States
(12) Patent Application Publication
(10) Pub. No.: US 2012/0016237 A1
(43) Pub. Date: Jan. 19, 2012

(54) ULTRASONIC DIAGNOSTIC APPARATUS, METHOD FOR CONTROLLING DISPLAY OF IMAGE AND CONTROL PROGRAM OF THE SAME

(76) Inventor: Shunichiro Tanigawa, Tokyo (JP)

(21) Appl. No.: 13/181,679

(22) Filed: Jul. 13, 2011

(30) Foreign Application Priority Data
Jul. 13, 2010 (JP) 2010-158869

(51) Int. Cl. A61B 8/00

(52) U.S. Cl. 600/438

(57) ABSTRACT

An ultrasonic diagnostic apparatus includes a physical amount calculation device configured to calculate a physical amount on elasticity of living tissue based on an echo signal obtained by transmitting an ultrasonic wave to the living tissue, and a display image control device configured to control display of a predetermined alternative elastic image displayed instead of an elastic image based on the physical amount calculated for error frames using a ratio of non-error frames or the error frames in a predetermined plurality of frames, wherein the error frames are determined as not meeting a standard.

### CROSS REFERENCE TO RELATED APPLICATIONS

This application claims the benefit of Japanese Patent Application No. 2010-158869 filed Jul. 13, 2010, which is hereby incorporated by reference in its entirety.

### BACKGROUND OF THE INVENTION

The embodiments described herein relate to an ultrasonic diagnostic apparatus and, more specifically, to an apparatus for displaying an elastic image indicating hardness or softness of living tissue and a control program of the same.

An ultrasonic diagnostic apparatus for displaying a combined image of a normal B-mode image and an elastic image indicating hardness or softness of living tissue is disclosed in Japan examined Patent 3932482 (to which US publication No. 2006/0052702A1 is related), for example. In this kind of ultrasound diagnostic apparatus, an elastic image is generated as follows. First, transmitting/receiving of an ultrasound wave is performed to obtain echo signals by repeating pressing and relaxing motion with an ultrasonic probe. Then, based on the obtained echo data, a physical amount of elasticity of the living tissue is calculated and the obtained physical amount is converted to hue information to generate a color elastic image. For the physical amount of elasticity of the living tissue, a strain of the living tissue is calculated, for example.

When the ultrasonic probe motion is changed from a pressing motion to a relaxing motion or otherwise, there are moments of no pressing and relaxing motions. Also, when it is operated by an unskilled operator, the degree of pressing or relaxing motion may be weak. This lack of degrees of pressing or relaxing motion causes insufficient deformation of the living tissue that the value calculated by the correlation calculation does not appears as differences corresponding to the difference in elasticity of the living tissue. In this case, the calculated physical amount is not an amount that accurately reflects the elasticity of living tissue.

Meanwhile, when the degree of pressing or relaxing motion by the probe is excessive, a transverse shift may occur at the living tissue. The echo signals obtained in such case contain noises due to the transverse shift and a correlation coefficient in a correlation calculation can be lower. Moreover, when the degree of pressing or relaxing motion of the probe is excessive, deformation of the living tissue may be larger so that the correlation windows set at two echo signals do not match and the correlation coefficient may be lower. If the correlation coefficient in the correlation calculation is lower, the physical amount accurately reflecting the elasticity of the living tissue cannot be obtained.

At regions having less ultrasonic reflectors or deep portions of the living tissue where transmitting/receiving ultrasonic does not reach due to attenuation, strength of the echo signals is insufficient. The correlation coefficient of the correlation calculation is lower for such echo data with insufficient strength. Also, when a direction of pressing or relaxing of the ultrasonic probe is not coincident with a direction of an acoustic ray of an ultrasonic wave, the above-mentioned transverse shift occurs so that the correlation coefficient of the correlation calculation becomes lower in the echo data obtained from such condition. Thus, the physical amount accurately reflecting the elasticity of the living tissue cannot be obtained.

As explained above, the elastic image generated based on the physical amount which the elasticity of the living tissue does not accurately reflect is not an image that reflects actual elasticity of the living tissue. Thus, there might be a possibility that the elasticity of the living tissue is not grasped accurately. Consequently, as Japan Unexamined Patent Publication No. 2010-99378 discloses, an ultrasonic diagnostic apparatus for displaying an alternative elastic image is provided. The alternative elastic image is generated by executing addition with weighting data of multiple frames after the weighting coefficient is set by frame based on reliability of the echo data.

However, it is inconvenient to display anytime the alternative elastic image generated by executing a weighted addition to multiple frames. For example, it is not appropriate to keep displaying the alternative elastic image generated by adding the data of frames in a condition that the ratio of error frames having lower reliability of echo signals is larger. Consequently, only under an appropriate condition, an ultrasonic diagnostic apparatus and a control program thereof that can display a predetermined alternative elastic image instead of an elastic image of error frames is desired.

### BRIEF DESCRIPTION OF THE INVENTION

A first aspect provides an ultrasonic diagnostic apparatus including: a physical amount calculation device for calculating a physical amount on elasticity of living tissue based on an echo signal obtained by transmitting an ultrasonic wave to the living tissue; a display image control device of controlling display or nondisplay of a predetermined alternative elastic image displayed instead of an elastic image based on the physical amount calculated for error frames, on the basis of a ratio of non-error frames or error frames in a predetermined plurality of frames, wherein the error frames are determined as they do not meet the standard.

According to a second aspect, in the ultrasonic diagnostic apparatus of the first aspect, the predetermined plurality of frames is most recent frames including a current frame.

According to a third aspect, in the ultrasonic diagnostic apparatus of the first aspect, the predetermined plurality of frames is most recent frames not including a current frame.

According to a fourth aspect, in the ultrasonic diagnostic apparatus according to any of preceding aspects, the predetermined alternative elastic image is an image obtained by adding an elastic image of a plurality of frames.

According to a fifth aspect, in the ultrasonic diagnostic apparatus according to any of preceding aspects, the determination device determines whether it is an error frame or not on the basis of a fact that an elastic image which is subject of determination is an image reflecting elasticity of living tissue accurately.

According to a sixth aspect, in the ultrasonic diagnostic apparatus of the fifth aspect, the physical amount calculation device sets correlation windows on an echo signal which is on the same acoustic ray but temporally different and calculates the physical amount by executing a correlation calculation between the correlation windows; the ultrasonic diagnostic apparatus further including: a physical amount average device for calculating an average of the physical amount for respective frames; and a comparison device configured to compare a calculated value obtained from the physical amount average device and an ideal value of the physical amount, wherein the determination device is further configured to execute the determination based on a result of the comparison device.

According to a seventh aspect, in the ultrasonic diagnostic apparatus of the fifth aspect, the physical amount calculation device sets correlation windows on an echo signal which is on the same acoustic ray but temporally different and calculates the physical amount by executing a correlation calculation between the correlation windows; the ultrasonic diagnostic apparatus further including: an correlation coefficient average device for calculating an average of a correlation coefficient in an correlation calculation between the correlation windows for respective frames; wherein the determination device executes the determination based on an average value obtained by the correlation coefficient average device.

According to a eighth aspect, in the ultrasonic diagnostic apparatus of the fifth aspect, the physical amount calculation device sets correlation windows on an echo signal which is on the same acoustic ray but temporally different and calculates the physical amount by executing a correlation calculation between the correlation windows; the ultrasonic diagnostic apparatus further including: an physical amount average device for calculating by frames an average of the physical amount obtained for correlation windows in which a correlation calculation of a correlation coefficient more than or equal to a predetermined threshold is executed; a ratio calculation device for calculating a ratio of a calculated value by the physical amount average device to an average amount of the physical amount which is pre-set; an correlation coefficient average device for calculating an average of a correlation coefficient in an correlation calculation between the correlation windows for respective frames; and a multiplication device for multiplying a calculated value of the ratio calculation device and a calculated value of the correlation coefficient average device; wherein the determination device executes the determination based on a calculated value by the multiplication device.

According to a ninth aspect, in the ultrasonic diagnostic apparatus of the fifth aspect, the physical amount calculation device sets correlation windows on an echo signal which is on the same acoustic ray but temporally different and calculates the physical amount with signs of plus and minus as the physical amount by executing a correlation calculation between the correlation windows; wherein, the determination device executes the determination based on a ratio of the plus and minus signs in one frame.

According to a tenth aspect, in the ultrasonic diagnostic apparatus of the fifth aspect, the determination device executes whether it is an error pixel or not on respective pixels and executes whether it is an error frame or not on the basis of a ratio of an error pixel or a non-error pixel in one frame.

According to a eleventh aspect, in the ultrasonic diagnostic apparatus of the tenth aspect, the determination device executes whether it is an error pixel or not on the basis of the physical amount calculated for respective pixels.

According to a twelfth aspect, in the ultrasonic diagnostic apparatus of the tenth aspect, the physical amount calculation device sets correlation windows on an echo signal which is on the same acoustic ray but temporally different and calculates the physical amount on respective pixels by executing the correlation calculation between the correlation windows; wherein, the determination device determines whether it is an error pixel or not on the basis of a correlation coefficient in the correlation calculation executed on respective pixels.

According to the thirteenth aspect, in the ultrasonic diagnostic apparatus according to any of preceding aspects, the display image control device displays, for a non-error frame, an elastic image based on the physical amount calculated on the non-error frame.

A fourteenth aspect provides a control program of an ultrasonic diagnostic apparatus for commanding a computer to execute functions. The program includes: a physical amount calculation function for calculating a physical amount on elasticity of living tissue based on an echo signal obtained by transmitting an ultrasonic wave to the living tissue; a display image control function, the display image control function controls display or nondisplay of a predetermined alternative elastic image displayed instead of an elastic image based on the physical amount calculated for an applicable error frame based a ratio of non-error frames or error frames in a predetermined plurality of frames, wherein error frames are determined as they do not meet the standard.

According to the above-mentioned aspects, displaying and hiding of the predetermined alternative elastic image displayed instead of the elastic image of the error frames determined that it is no fulfilled a predetermined standard is controlled on the basis of the ratio of non-error frames in a predetermined frames or the ratio of error frames. So, the alternative elastic image can be displayed only under an appropriate condition.

Further objects and advantages of the embodiments described herein will be apparent from the following description of embodiments of the invention as illustrated in the accompanying drawings.

### BRIEF DESCRIPTION OF THE DRAWINGS

FIG. 1 is a block diagram showing one example of a schematic configuration of embodiments of the ultrasonic diagnostic apparatus.

FIG. 2 is an explanation drawing for calculating of strains.

FIG. 3 is an explanation drawing for generating a B-mode image data and an elastic image data.

FIG. 4 is a block diagram showing the configuration of the display control device in the ultrasonic diagnostic apparatus in the first embodiment.

FIG. 5 is a figure showing one example of display of the display device in the ultrasonic diagnostic apparatus shown in FIG. 1.

FIG. 6 is a figure showing another example of display of the display device in the ultrasonic diagnostic apparatus shown in FIG. 1.

FIG. 7 is a figure showing another example of display of the display device in the ultrasonic diagnostic apparatus shown in FIG. 1.

FIG. 8 is a figure showing a graph of function used at the ratio calculation device.

FIG. 9 is a flow chart showing an operation of the embodiment of the ultrasonic diagnostic apparatus.

FIG. 10 is a figure for explaining the determination by the ratio determination device in the step S3 in FIG. 8.

FIG. 11 is a figure for explaining the determination by the ratio determination device in the step S3 and display of the ultrasonic image in the step S4, S5 in FIG. 8.

FIG. 12 is a figure for explaining the determination by the ratio determination device in the step S3 and display of the ultrasonic image in the step S4, S5 in FIG. 8.

FIG. 13 is a figure for explaining the determination by the ratio determination device in the step S3 and display of the ultrasonic image in the step S4, S5 in FIG. 8.

FIG. 14 is a block diagram showing the configuration of the display control device in the second embodiment.

FIG. 15 is a block diagram showing the configuration of the display control device in the third embodiment.

FIG. 16 is a block diagram showing the configuration of the display control device in the fourth embodiment.

FIG. 17 is a block diagram showing the configuration of the display control device in the fifth embodiment.

### DETAILED DESCRIPTION OF THE INVENTION

The embodiments of the invention will be described in detail based on the figures.

### First Embodiment

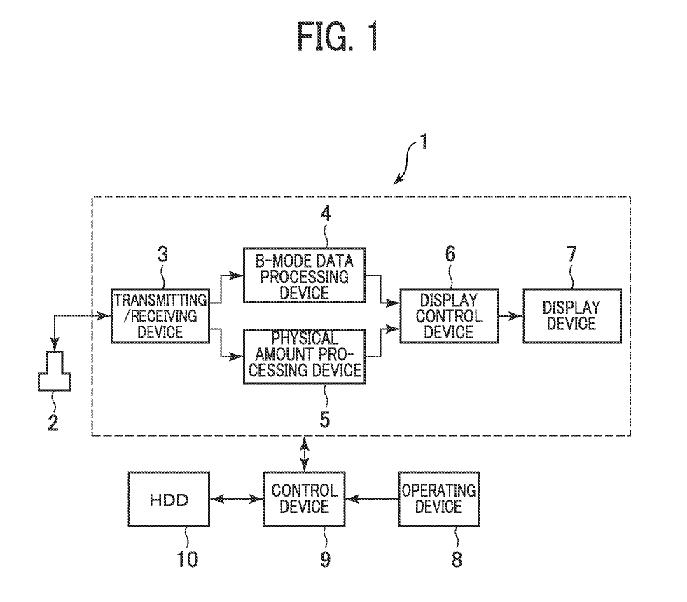

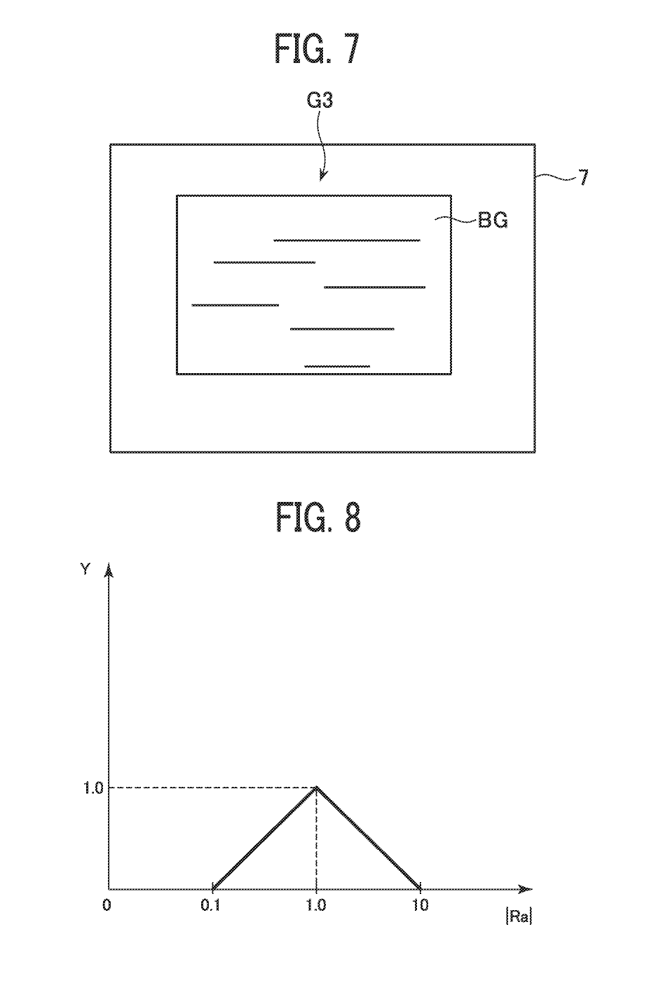

First of all, the first embodiment will be explained based on FIG. 1 through FIG. 13. An ultrasonic diagnostic apparatus 1 shown in FIG. 1 includes an ultrasonic probe 2, a transmitting/receiving device 3, a B-mode data processing device 4, a physical amount processing device 5, a display control device 6, a display device 7, an operating device 8, a control device 9, and a HDD (Hard Disk Drive) 10.

The ultrasonic probe 2 transmits an ultrasonic wave to living tissue and receives an echo signal. Under the condition that the ultrasonic probe 2 is in contact with a surface of the living tissue, pressing and relaxing motions of the ultrasonic probe 2 is repeated and the ultrasonic probe 2 applies an acoustic radiation pressure to the living tissue. As a result, the ultrasonic probe 2 obtains an echo data with executing transmitting/receiving of an ultrasonic wave, deforming the living tissue. Based on the obtained echo data, an elastic image is generated as described below.

The transmitting/receiving device 3 drives the ultrasonic probe 2 under a predetermined scanning condition and scans an ultrasonic wave of each acoustic ray. Also, it executes signal processing, such as a phasing-adding process, on the echo data received by the ultrasonic probe 2. The echo signal signal-processed at the transmitting/receiving device 3 is output to the B-mode data processing device 4 and the physical amount processing device 5.

The B-mode data processing device 4 executes a B-mode process, such as a logarithmic compression process or an envelope detection process, to the echo data output from the transmitting/receiving device 3 to generate B-mode data. The B-mode data is output from the B-mode data processing device 4 to the display control device 6.

The physical amount processing device 5 generates a physical amount data calculating the physical amount on the elasticity at respective members in the living tissue based on the echo data output from the transmitting/receiving device 3 (physical amount calculating function). As disclosed in US Publication No. 2008/0119732 A1, the physical amount processing device 5 sets correlate windows to temporally different echo data on the same acoustic ray in one scanning surface, calculates the physical amount on the elasticity pixel by pixel after executing the correlation calculation in between the correlation windows, and generates the physical amount data for one frame. The physical amount processing device 5 calculates a strain St as a physical amount on the elasticity in this embodiment. The physical amount processing device 5 is one example of the embodiments of the physical amount processing device in the invention and also one example of the embodiments of the physical amount calculation function.

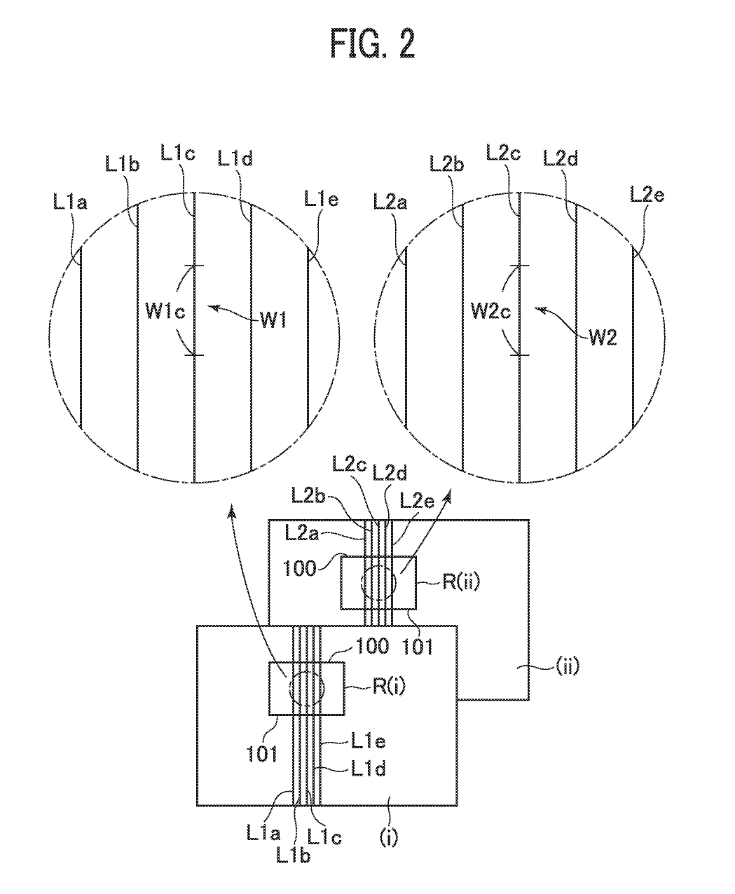

One example for calculating the strain St is explained in detail. The physical amount processing device 5 sets correlation windows to respective echo signals belonged to a frame (i), (ii), as shown in FIG. 2. Particularly, the physical amount processing device 5 sets a correlation window W1 to an echo signal belonged to the frame (i) and a correlation window W2 to an echo signal belonged to the frame (ii). Those correlation windows W1, W2 correspond to one pixel. Then, the physical amount processing device 5 executes the correlation calculation between the correlation windows W1 and W2 and calculates the strain St.

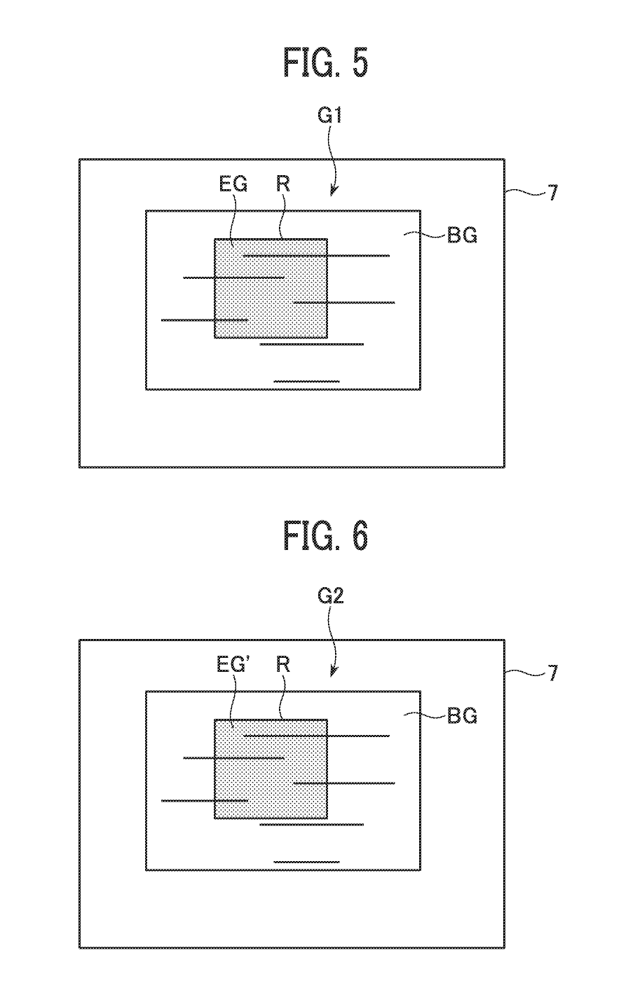

In FIG. 2, the frames (i), (ii) include the echo signals obtained on multiple acoustic rays. In FIG. 2, five acoustic rays L1a, L1b, L1c, L1d, L1e are shown as a part of the multiple acoustic rays. Also, as acoustic rays corresponding to the acoustic rays L1a through L1e, acoustic rays L2a, L2b, L2c, L2d, L2e are shown. That is, the acoustic rays L1a and L2a, the acoustic rays L1b and L2b, the acoustic rays L1c and L2c, the acoustic rays L1d and L2d, and the acoustic rays L1e and L2e belong to different two frames and correspond to temporally-different acoustic ray. Further in FIG. 2, R (i), R (ii) indicate regions corresponding to elastic image display regions R (see FIG. 5 and FIG. 6), which will be explained later.

For example, the correlation window W1c is set on the echo signal of the acoustic ray L1c as the correlation window W1, and the correlation window W2c is set on the echo signal of the acoustic ray L2c as the correlation window W2. The physical amount processing device 5 executes the correlation calculation between the correlation windows W1c, W2c to calculate the strain St. The physical amount processing device 5 sets sequentially the correlation windows W1c, W2c from the top end 100 to the bottom end 101 of the regions R (i), R (ii). The physical amount processing device 5 also calculates the strain St of other acoustic rays in the regions R(i), R(ii) similarly.

The strain St calculated by the physical amount processing device 5 is calculated with plus and minus signs corresponding to directions that the living tissue deforms to. For example, a displacement of minus sign is calculated mainly when it is a direction that the living tissue is pressed, and conversely a displacement of plus sign is calculated mainly when it is a direction that the living tissue returns to the original formation.

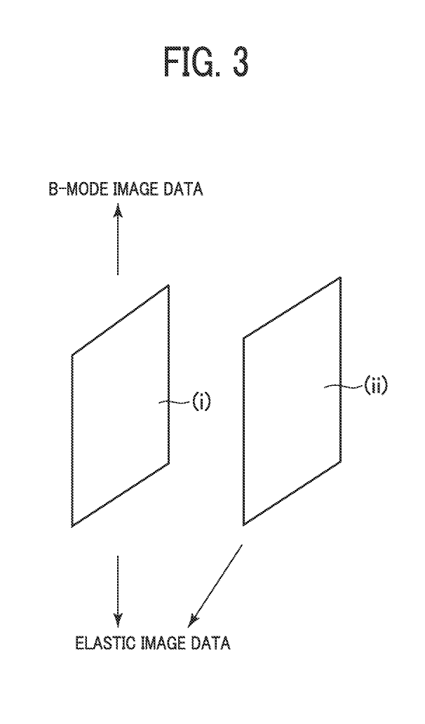

As FIG. 3 shows, an elastic image that will be discussed later is generated for one frame from the echo signals belonged to different two frames (i), (ii). Meanwhile, the B-mode image data that will be discussed later is generated from either of the echo signals of the frame (i), (ii).

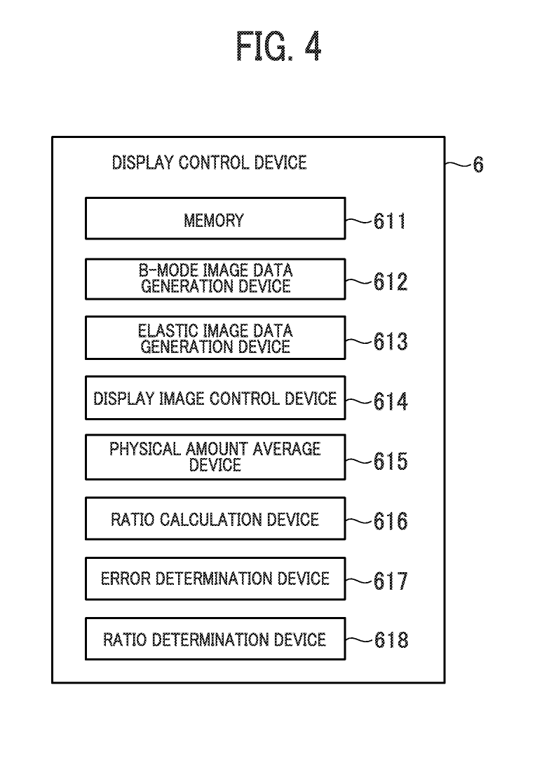

The B-mode data from the B-mode data processing device 4 and the physical amount data from the physical amount processing device 5 are input to the display control device 6. The display control device 6 includes, as shown in FIG. 4, a memory 611, a B-mode image data generation device 612, an elastic image data generation device 613, a display image control device 614, a physical amount average device 615, a ratio calculation device 616, an error determination device 617, and a ratio determination device 618.

The memory 611 stores the B-mode data and the physical amount data. The B-mode data and the physical amount data are stored in the memory 611 as data of respective acoustic rays. As described later, the B-mode data before being scan-converted to the B-mode image data by a scan converter and the physical amount data before being scan-converted to the elastic image data are referred as “raw data”.

The memory 611 includes a semiconductor memory, such as RAM (Random Access Memory) or ROM (Read Only Memory). Note that the B-mode data and the physical amount data can be stored in the HDD 10.

The B-mode image data generation device 612 executes a scan conversion with a scan converter on the B-mode data to convert the B-mode data to B-mode image data having brightness information in response to the signal strength of the echo signal. The B-mode image data has brightness information of 256 tones, for example.

The elastic image data generation device 613 executes a scan conversion with a scan converter to convert the physical amount data to a color elastic image data having hue information in response to the strain. The color elastic image data has 256 tones of hue information, for example.

The display image control device 614 executes a display image control function and displays any one of ultrasonic images of ultrasonic images G1, G2, or G3 shown in FIG. 5 through FIG. 7. As described later, for the non-error frames the display image control device 614 combines the B-mode image data and the color elastic image data by calculating both data and generates an image data of ultrasonic image G1 to be displayed on the display device 7. The display image control device 614 displays the image data, as shown in FIG. 5, on the display device 7 as an ultrasonic image G1 that is combined image of the black-and-white B-mode image BG and the color elastic image EG. That is, for the non-error frame, the elastic image EG and the B-mode image BG based on the strain St calculated for the non-error frame are displayed. The elastic image EG is displayed semi-translucent (in a condition that the background of B-mode is translucent) within the elastic image display region R set on the B-mode image BG.

Further for the error frame, the display image control device 614 displays an ultrasonic image G2 that is a combined image of a predetermined alternative elastic image EG′ and the B-mode image BG, as shown in FIG. 6, instead of the elastic image EG based on the strain St calculated for the error frame, or displays an ultrasonic image G3 comprised of only B-mode image BG without displaying the elastic image EG and the alternative elastic image EG′ (display image control function). The details will be explained later. The display image control device 614 is one example of the embodiments of the display image control device, and the display image control function is one example of the embodiment of the display image control function.

The physical amount average device 615 calculates an average value RSt_{AV} of the strain in the elastic image EG with respect to each frame. In particular, the physical amount average device 615 calculates the average value RSt_{AV} of the strain calculated for respective pixels in the elastic image display region R that displays the elastic image EG. Note that because the strain St may be minus, or negative, there is a possibility that the average value RSt_{AV} can be minus, or negative. The physical amount average device 615 is one example of the embodiments of the physical amount average device.

However, in the elastic image display region R (the region R(i), R(ii)), the physical amount average device 615 may calculate the average value RSt_{AV} of the strain St of the pixels where the correlation coefficient C (0≦C≦1) in a correlation calculation for calculating the strain St is more than the predetermined value.

The ratio calculation device 616 calculates a ratio Ra=RSt_{AV}/ISt_{AV} of the average value RSt_{AV} to an ideal value ISt_{AV} of an average of the strain, and further executes a calculation with Equation 1 to calculate a calculated value Y for frame by frame.

Y=1.0−|log₁₀|Ra|| (Eq. 1)

The ratio calculation device 616 is one example of the embodiments of the comparison device and the ratio calculation devices. The ideal value ISt_{AV} is one example of the embodiments of the ideal value. The calculated value Y is one example of the embodiments of comparison result of the comparison device and calculated values of the ratio calculation device.

The ideal value ISt_{AV} is explained here. When the degree of deformation of the living tissue is too small, an elastic image that accurately reflects the elasticity of the living tissue cannot be obtained. Also, when the living tissue is deformed by pressing and relaxing of the ultrasonic probe 2 to the living tissue, a transverse shift may occur if the degree of pressing and relaxing is excessive. So, the elastic image based on the echo signal obtained in the condition is not an image that reflects the elasticity of the living tissue accurately. Therefore, it is necessary to deform the living tissue moderately in order to obtain the elastic image that reflects the deformation of living tissue accurately. The ideal value ISt_{AV} is an average value of the strain St obtained in a region set optionally when transmitting/receiving of an ultrasonic wave is performed to the living tissue which is moderately deformed allowing obtaining an elastic image which reflects the elasticity of the living tissue accurately. This ideal value ISt_{AV} is a value obtained empirically after performing an experiment on phantom having same hardness of tumor or normal tissue equally to an actual living tissue, for example. Further the ideal value ISt_{AV} can be configurable by an operator at the operating device 8, or can be stored in an apparatus as default.

For explanation of Equation 1, Equation 1 is for making the ratio Ra to be within a range between 0 to 1 and the Y obtained by Equation 1 is equal to the ratio of the average value RSt_{AV} to the ideal value ISt_{AV}. When a function expressed in Equation 1 is expressed in a graph, the graph is the one shown in FIG. 8. As shown in FIG. 8, the function is 0≦Y≦1.

It is assumed that 0.1≦|Ra|≦10 and if |Ra| excesses this range, the Y is zero.

A calculated value Y of the ratio calculation device 616 is a value indicating quality of the elastic mage, and the elastic image can be known how the elasticity of the living tissue is reflected accurately in the image based on the calculated value Y. In particular, as the calculated value Y is near to 1, the quality of elastic image is in a “good quality”, on the other hand as the calculated value Y is close to zero, the quality of elastic image is in a “bad quality”. “Good quality” means that an elastic image accurately reflects the elasticity of the living tissue, and on the other hand, “bad quality” means that the elastic image does not reflect the elasticity of the living tissue accurately.

For more detailed explanation about the relationship between the calculated value Y and the quality of elastic image, as understood from FIG. 8, when the average value RSt_{AV} is equal to ISt_{AV}, (which is |Ra| is 1), the calculated value Y is 1. Therefore, if the calculated value Y is 1 or a value close to 1, the deformation of the living tissue is moderate and it can be recognized that an elastic image reflecting the elasticity of the living tissue is obtained accurately.

Meanwhile, when the average value RSt_{AV} is a value far from the ideal value ISt_{AV} (that is, |Ra| is as far value as from 1), the calculated value Y becomes closer to zero. Here, the average value RSt_{AV} being farther from the ideal value ISt_{AV} means that degree of deformation of the living tissue is not appropriate. Thus, as the calculated value Y becomes closer to zero, the deformation of the living tissue is not appropriate and that means the elastic image reflecting the elasticity of the living tissue accurately is not obtained.

The error determination device 617 determines whether it is an error frame or not. The error determination device 617 determines whether it is an error frame or not based on an aspect that if the echo signal in respective frames can obtain the elastic image reflecting the elasticity of the living tissue accurately. The error determination device 617 is one example of the embodiments of a determination device.

Based on the calculated value Y described above, the elastic image is understood how accurately the image reflects the elasticity of the living tissue. Thus, in this embodiment, the error determination device 617 determines if it is an error frame or not based on the calculated value Y.

The ratio determination device 618 calculates a ratio of non-error frames in predetermined frames and determines whether it is more than a predetermined ratio or not. The detail will be explained later.

The display device 7 includes, for example, LCD (Liquid Crystal Display) or CRT (Cathode Ray Tube). The operating device 8 includes a keyboard and a pointing device (not shown) for inputting a command or information by an operator.

The control device 9 includes CPU (Central Processing Unit), and reads out a control program stored in the HDD 10 and executes functions in respective members of the ultrasonic diagnostic apparatus 1, like the physical amount calculating function and the displaying image control function.

The operation of the ultrasonic diagnostic apparatus 1 in the present embodiment is explained here. First of all, the transmitting/receiving device 3 transmits an ultrasonic wave to living tissue of the subject through the ultrasonic probe 2 and obtains an echo signal. In this operation, transmitting/receiving of the ultrasonic wave is performed by deforming the living tissue. Methods to deform living tissue are, for example, a method of repeating pressing and relaxing to the subject by the ultrasonic probe 2 or a method of applying acoustic radiation pressure to the subject through the ultrasonic probe 2.

After the echo signal is obtained, the B-mode data processing device 4 generates the B-mode data, and the physical amount data processing device 5 generates the physical amount data. Further, the B-mode image data generation device 612 generates the B-mode image data and the elastic image data generation device 613 generates the color elastic image data. Then, the display image control device 614 displays any one of ultrasonic images G1 through G3 on the display device 7.

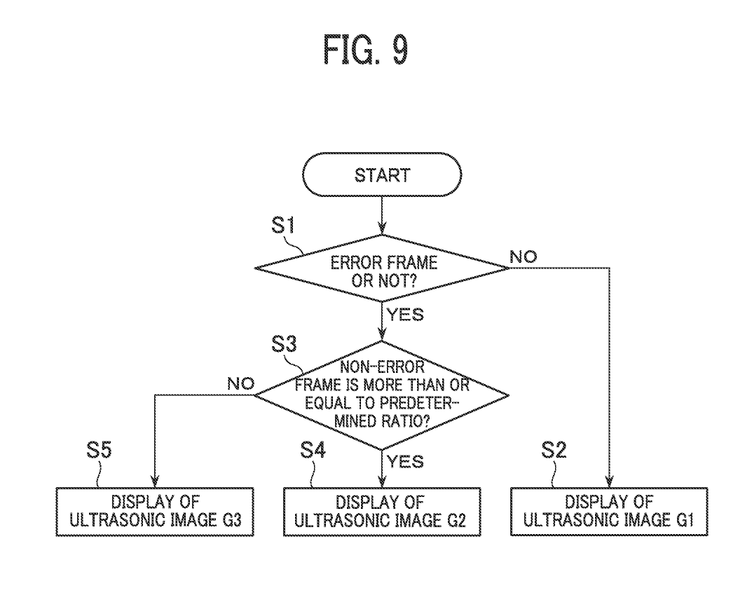

The display of ultrasonic images is explained referring to the flow chart in FIG. 9. In the ultrasonic diagnostic apparatus 1, the process shown in FIG. 9 is executed for each frame and any one of the ultrasonic images G1 through G3 is displayed. In particular, firstly in step S1, the error determination device 617 determines whether it is an error frame or not based on the calculated value Y. More particularly, the error determination device 617 determines a frame as an error frame when the calculated value Y is less than or equal to a threshold value Y_{TH}.

Regarding the threshold value Y_{TH}, the threshold value Y_{TH} is set to a value that expresses elasticity of the living tissue accurately to some extent. Because 0≦Y≦1, the threshold value Y_{TH} is also set within a range more than or equal to 0 and less than or equal to 1. The threshold value Y_{TH} can be stored in the HDD 10 in advance or can be set by an operator through the operating device 8.

When it is not determined as an error frame in the step S1 (NO in step S1), it goes on to step S2 process. Meanwhile, when it is determined as an error frame in the step S1 (YES in step S1), it goes on to step S3 process.

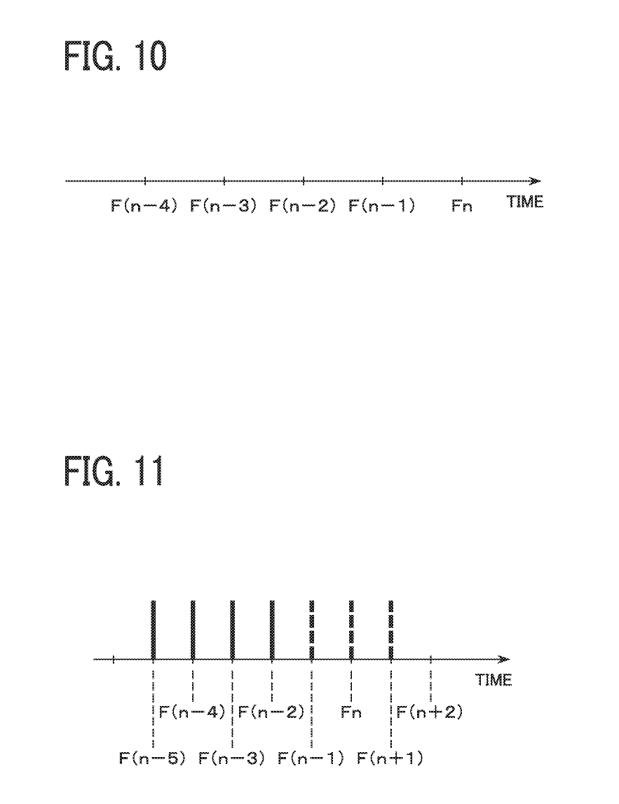

In the step S2, the display image control device 614 displays the ultrasonic image G1. On the other hand, in the step S3, the ratio determination device 618 can calculate a ratio of non-error frames or error frames in multiple frames formed by the most recent frames and the current frame Fn, in other words, the current frame Fn and previous predetermined frames backing from the current frame Fn by predetermined number of frames. The non-error frame is a frame in which the calculated value Y is exceeded the threshold value Y_{TH}. For example, the ratio determination device 618 calculates a ratio of non-error frames in the current frame Fn and four frames back from the current frame Fn, F (n-1), F(n-2), F(n-3), F(n-4), which means five frames in total, as predetermined frames shown in FIG. 10. Then it determines whether or not the ratio of non-error frames is more than or equal to “m” of five (“m” is any one of 2, 3, or 4).

In the step S3, the ratio of non-error frames is determined as more than or equal to the predetermined ratio (YES in step S3), it goes on to step S4 process. On the other hand, the ratio of non-error frames is determined as less than the predetermined ratio (NO in step S3), it goes on to step S5 process. In the step S4, the display image control device 614 displays an ultrasonic image G2 that is combined of a predetermined alternative image EG′ and a B-mode image BG. Meanwhile, in the step S5, the display image control device 614 displays an ultrasonic image G3 including only the B-mode image BG.

The predetermined alternative elastic image EG′ is explained here. The alternative elastic image EG′ is an image based on the data obtained by executing weighting addition of the color elastic image data of the multiple frames. The weighting addition processing can be executed on the most recent multiple frames including the current frame Fn or on the most recent multiple frames without the current frame Fn. Regarding the weighting addition processing, the weighting coefficient is preferably set lower than the non-error frame.

In particular, determination by the ratio determination device 618 in the step S3 and display of the ultrasonic images G2, G3 in the step S4, S5 are explained referring FIG. 11 through FIG. 13. In FIG. 11 through FIG. 13, the frames on which solid lines are drawn are non-error frames and means that they are frames on which the ultrasonic image G1 combined of the elastic image EG and the B-mode image BG of relevant frames are displayed. The frames on which dashed lines are drawn are error frames and means they are frames on which the ultrasonic image G2 combined of the alternative elastic image EG′ and the B-mode image BG are displayed instead of the elastic image EG based on the color elastic image data of the relevant frame. The frames without lines are error frames and means they are frames on which the ultrasonic image G3 including only the B-mode image (a frame on which the elastic image is not displayed) are displayed.

The ratio determination device 618 determines whether or not the ratio of non-error frames is more than or equal to two-fifths, and if it is more than or equal to two-fifths, it goes on to step S4 process and if it is less than two-fifths, it goes on to step S5.

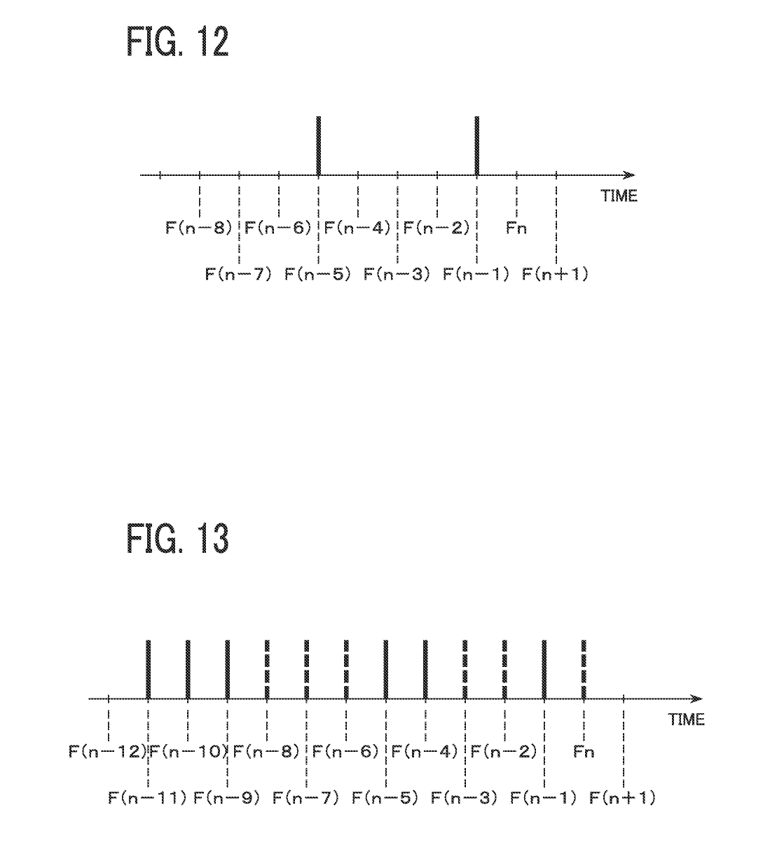

In FIG. 11, the ratio of non-error frames among the frame Fn, F(n-1), F(n-2), F(n-3), F(n-4) is three-fifths. Thus, the method goes on to step S4 process and the ultrasonic image G2 displaying the alternative elastic image EG′ are displayed. In FIG. 12, the ratio of non-error frames among the frame Fn, F(n-1), F(n-2), F(n-3), F(n-4) is one-fifth. Thus, the method goes on to step S5 process and the ultrasonic image G3 including only the B-mode image BG is displayed. In FIG. 13, the ratio of non-error frames among the frame Fn, F(n-1), F(n-2), F(n-3), F(n-4) is three-fifths. Thus, the method goes on to step S4 process and the ultrasonic image G2 displaying the alternative elastic image EG′ is displayed.

FIG. 11 through FIG. 13 is explained in more detail. First in FIG. 11, frames F (n+1), F (n+2) are error frames. The ratio of non-error frames among the frames F (n+1), Fn, F (n-1), F (n-2), F (n-3) is two-fifths, and the ultrasonic image G2 is displayed on the frame F (n+1). Meanwhile, the ratio of non-error frames among F (n+2), F (n+1), Fn, F (n-1), F (n-2) is one-fifth and the ultrasonic image G3 is displayed on the frame F (n+2). As shown in FIG. 11, in the case frames after the frame F (n-1) become error frames despite that through frames F (n-5) to F (n-2) are non-error frames, the alternative elastic image EG′ is displayed partway, but it is not displayed from the certain point.

There is a request to search for the lesion location by observing only a B-mode image when screening is performed to search for a lesion location, such as a tumor, by changing scanning position while displaying the ultrasonic images. Thus, if an operator stops pressing and relaxing actions by the ultrasonic probe 2 at screening, the calculated value Y becomes lower than the threshold value Y_{TH}. So, the error frames like frames after the frame F (n-1) as shown in FIG. 11 continue displaying the ultrasonic image G3 including only the B-mode image automatically. Therefore, the alternative elastic image EG′ can be displayed only in a condition where the ratio of non-error frames is more than or equal to the predetermined ratio.

Next in FIG. 12, frames F (n-4) through F (n-2) are error frames. The ratio of non-error frames among frames F (n-4), F (n-5), F (n-6), F (n-7), F (n-8) is one-fifth, and the ultrasonic image G3 is displayed on the frame F (n-4). The ratio of non-error frames among frames F (n-3), F (n-4), F (n-5), F (n-6), F (n-7) is one-fifth, and the ultrasonic image G3 is displayed on the frame F (n-3). The ratio of non-error frames among frames F (n-2), F (n-3), F (n-4), F (n-5), F (n-6), is one-fifth, and the ultrasonic image G3 is displayed on the frame F (n-3). As shown in

FIG. 12, when the frames are non-error frames at interval, it may be caused by executing pressing or relaxing action with the ultrasonic probe 2 unconsciously by an operator. Even in such condition, the ultrasonic image G3 only including the B-mode image BG can be displayed so that there is a low possibility of interfering with the screening. As pointed out in the above-mentioned explanation, the alternative elastic image EG′ can be displayed only in an appropriate condition where the ratio of non-error frames is more than or equal to the designated ratio.

Next in FIG. 13, frames F (n-8) through F (n-6), F (n-3), F (n-2), F (n+1) are error frames. The ratio of non-error frames among the frames F (n-8) through F (n-6), F (n-3), F (n-2) is two-fifths, and the ultrasonic image G2 is displayed. Meanwhile, the ratio of non-error frames in the frame F (n+1) is one-fifth and the ultrasonic image G3 is displayed. As shown in FIG. 13, when the ratio of error frames increases gradually, it can be considered as a moment between the condition executing pressing and relaxing actions by the ultrasonic probe 2 while displaying the elastic image and the screening step. In this condition, when the ratio of non-error frames is more than or equal to the predetermined ratio, the alternative elastic image EG′ is displayed, but when the ratio of non-error frames becomes less than the predetermined ratio, only B-mode image BG is displayed so that there is a low possibility of interfering with the screening. Thus, the alternative elastic image EG′ can be displayed only in an appropriate condition.

According to the above-mentioned embodiment, in the condition that the ratio of error frames is more than or equal to the predetermined ratio, the alternative elastic image EG′ is displayed for the error frames so that the elastic image which reflects the elasticity of actual living tissue as actual as possible is displayed. Meanwhile, when the ratio of error frames is less than the predetermined ratio, only the B-mode image BG is displayed so that continuous display of the alternative elastic image EG′ can be prevented if the ratio of error frames is increased. Therefore, the alternative elastic image EG′ can be displayed only in an appropriate condition.

### Second Embodiment

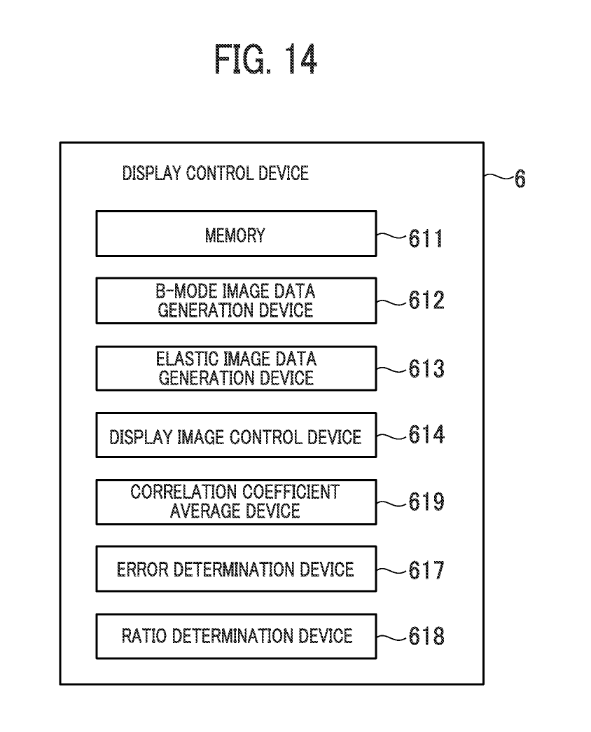

Next, the second embodiment is explained based on FIG. 14. Note that the same components as the first embodiment have the same numberings and explanation will be omitted.

In this embodiment, the display control device 6 includes the memory 611, the B-mode image data generation device 612, the elastic image data generation device 613, the display image control device 614, the error determination device 617, the ratio determination device 618, and a correlation coefficient average device 619. The correlation coefficient average device 619 is one example of the embodiments of a correlation coefficient average device.

The correlation coefficient average device 619 calculates an average value C_{AV} for each frame in the elastic image display region R (the region R(i), R(ii)) of a correlation coefficient C in the correlation calculation for each pixel executed by the physical amount data processing device 5. Here, it is 0≦C≦1 so that 0≦C_{AV}≦1. As the correlation coefficient in the correlation calculation is close to 1, displacement reflecting the elasticity of living tissue accurately can be obtained, on the other hand, as the correlation coefficient becomes zero, displacement reflecting the elasticity of living tissue accurately cannot be obtained. Therefore, as the average value C_{AV} is close to 1, quality of the elastic image EG is in a good quality while as the average value C_{AV} is close to zero, quality of the elastic image EG is in a bad quality.

In the embodiment, in the step S1 shown in FIG. 9, the error determination device 617 determines whether the frame is an error frame or not on the basis of the average value C_{AV} of the correlation coefficient C. The error determination device 617 determines as an error frame when the average value C_{AV} is less than or equal to the threshold value C_{TH}.

By the correlation coefficient C mentioned above, the elastic image can be understood how accurately the elastic image reflects the elasticity of living tissue. Thus in this embodiment, the error determination device 617 determines whether the frame is an error frame or not on the basis of the average value C_{AV} of the correlation coefficient C.

In the elastic image of the frame having the average value C_{AV} that is over the threshold value C_{TH}, the threshold value C_{TH} is set to a value indicating the elasticity of living tissue accurately to an extent.

The second embodiment explained above can obtain the same effect of the first embodiment.

### Third Embodiment

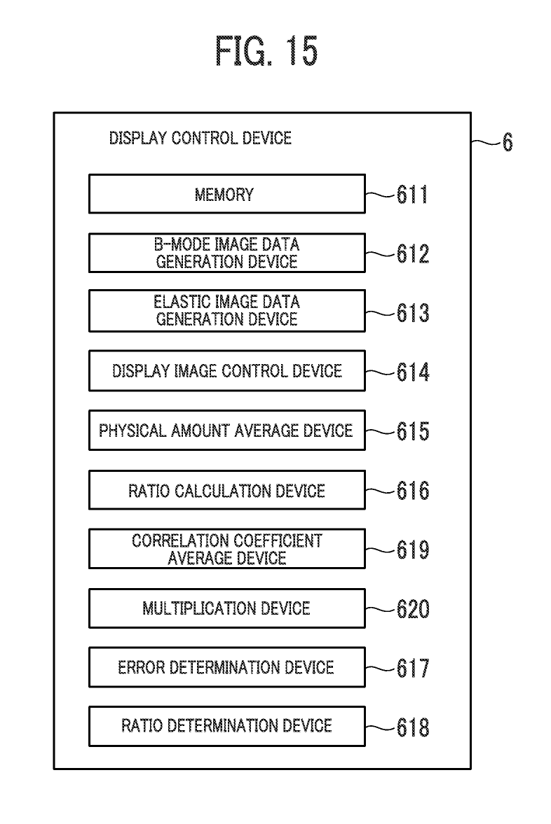

Next, the third embodiment is explained based on FIG. 15. Note that the same components as the first and second embodiments have the same numberings and explanation will be omitted.

In this embodiment, the display control device 6 includes the memory 611, the B-mode image data generation device 612, the elastic image data generation device 613, the display image control device 614, the physical amount average device 615, the ratio calculation device 616, the error determination device 617, the ratio determination device 618, the correlation coefficient average device 619, and a multiplication device 620 additionally. The multiplication device 620 is one example of the embodiments of a multiplication device.

In this embodiment, the physical amount average device 615, in the elastic image display region R (the region R (i), and the region R (ii)), calculates the average value RSt_{AV′} of the strain St of the pixel (correlation window) on which the correlation calculation that the correlation coefficient C is more than or equal to the designated value is executed. Then, the ratio calculation device 616 calculates the ratio Ra using the average value RSt_{AV′} instead of the average value RSt_{AV} and calculates the calculated value Y from Equation 1. The correlation coefficient average device 619 calculates the average value C_{AV} of the correlation coefficient C similarly to the second embodiment.

The multiplication device 620 multiplies the calculated value Y obtained by the ratio calculation device 616 and the average value C_{AV} of the correlation coefficient C obtained by the correlation coefficient average device 619 and calculates the multiplication value M. The multiplication value M is calculated with each frame.

The multiplication device 620 can weight and multiply when the calculated value Y and the average value CAV of the correlation coefficient C are multiplied.

Here, 0≦Y≦1, 0≦C_{AV}1 so that 0≦M≦1. Because the multiplication value M is a multiplication value of the calculated value Y and the average value C_{AV} so that the quality of elastic image EG becomes in a good quality as the multiplication value M is close to 1, while the quality of elastic image EG becomes in a bad quality as the multiplication value M is close to 0.

In this embodiment, in the step S1 shown in FIG. 9, the error determination device 617 determines whether the frame is an error frame or not based on the multiplication value M. The error determination device 617 determines the error frame when the multiplication value M is less than or equal to the threshold value M_{TH}.

Based on the calculated value Y and the correlation coefficient C, it is determined how much the elastic image reflects the elasticity of the living tissue accurately so that it is also determined how much the elastic image reflects the elasticity of the living tissue accurately based on the multiplication value M. Thus, in this embodiment, the error determination device 617 determines whether it is the error frame or not based on the multiplication value M.

Regarding the threshold value M _{TH}, in the elastic image of frame having the multiplication value M which is over the threshold value M _{TH}, the threshold value M _{TH}, is set to a value indicating the elasticity of the living tissue accurately to some extent.

In the third embodiment explained above can obtain the same effect of the first and second embodiments.

### Fourth Embodiment

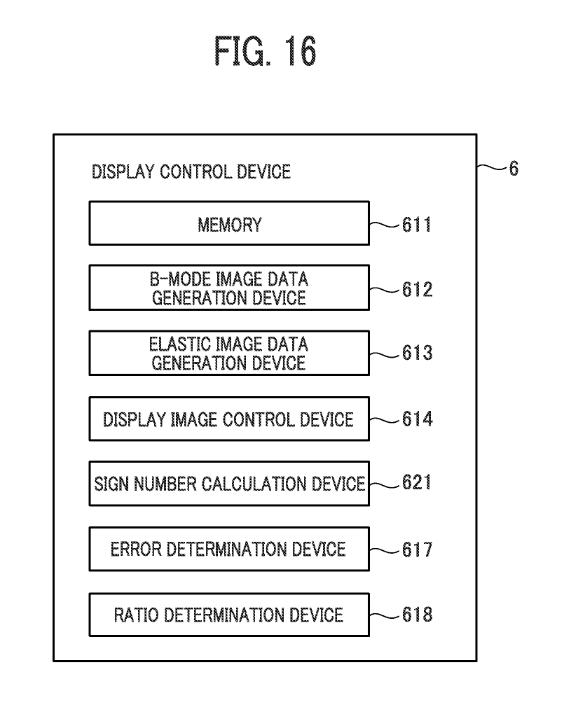

Next, the fourth embodiment is explained based on FIG. 16. Note that the same components as the first through third embodiments have the same numberings and explanation will be omitted.

In this embodiment, the display control device 6 includes the memory 611, the B-mode image data generation device 612, the elastic image data generation device 613, he display image control device 614, the error determination device 617, the ratio determination device 618, and a sign number calculation device 621 additionally. The sign number calculation device 621 finds number of plus signs and number of minus signs for the strain St calculated on each pixel in one frame.

In this embodiment, in the step S1 shown in FIG. 9, the error determination device 617 determines whether it is the error frame or not on the basis of the ratio of the number of plus signs and the number of minus signs. In particular, if the condition of Equation 2 or Equation 3 is fulfilled, the frame is determined as the error frame, meanwhile, if the conditions of Equation 2 and Equation 3 is not fulfilled, the frame is determined as the error frame.

Number of plus signs>x×number of minus signs (Eq. 2)

Number of minus signs>x×number of plus signs (Eq. 3)

In Equation 2 and Equation 3, x≦1. The “x” can be input to the operating device 8 in advance by an operator or can be stored in the HDD 10 in advance.

The relationship of the ratio of a sign of the strain St in one frame and the quality of elastic image EG is explained here. For example, if the pressing and relaxing actions by the ultrasonic probe 2 are performed appropriately, the ratio of either positive or negative becomes larger as the ratio of the sign of the strain St in one frame. However, if the pressing and relaxing actions by the ultrasonic probe 2 are not performed appropriately and transverse shift of the living tissue is occurred, the ratio of the sign does not slant to either positive or negative and the ratio of both signs are balanced as the ratio of the sign of the strain St in one frame. Thus, based on the ratio of positive and negative signs, it can be determined how much the elastic image reflects the elasticity of the living tissue accurately. From the above-mentioned explanation, if any condition of Equation 2 and Equation 3 is fulfilled, the ratio of the positive and negative signs is balanced so that the frame is determined as the error frame.

In the fourth embodiment explained above can obtain the same effect of the first through third embodiments.

### Fifth Embodiment

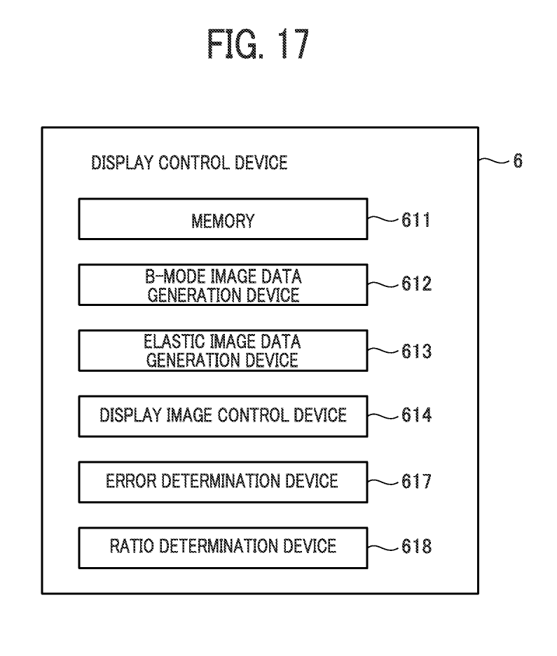

Next, the fifth embodiment is explained based on FIG. 17. Note that the same components as the first through fourth embodiments have the same numberings and explanation will be omitted.

In this embodiment, the display control device 6 includes the memory 611, the B-mode image data generation device 612, the elastic image data generation device 613, the display image control device 614, the error determination device 617, and the ratio determination device 618.

In this embodiment, in the step S1 of FIG. 9, the error determination device 617 is different from the first through fourth embodiments and firstly determines whether it is the error pixel or not about each pixel in the elastic image display region (the region R (i), R (ii)) of one frame. Then, it determines whether it is the error frame or not on the basis of the ratio of pixel determined as an error (error pixel) and of pixel not determined as an error (non-error pixel).

In this embodiment, the error determination device 617 determines whether it is an error pixel or not on the basis of the respective strains St calculated by respective pixels. For example, the error determination device 617 determines the pixel as the error pixel when the strain St is not within the pre-set predetermined range. Or, the error determination device 617 can determine whether it is the error pixel or not to respective pixels on the basis of the statistical distribution of the strain St within the elastic image display region R (the region R (i), the region R (ii)). As just described, in the case that determination is executed based on the statistic distribution of the strain St, for example, in the statistic distribution of the strain St, the calculated pixels to which the strain “p” percents in a high order or in “p” percents in a low order belong can be determined as an error. The “p” can be designed arbitrarily.

In the fifth embodiment explained above can obtain the same effect of the first through fourth embodiments.

Next, a variation of the fifth embodiment is explained. In this variation embodiment, the error determination device 617 determines whether it is an error pixel or not on the basis of the correlation coefficient C in the correlation calculation executed on each pixel. For example, the error determination device 617 determines the pixel as the error pixel when the correlation coefficient C is less than or equal to the predetermined value.

The invention was explained with above-mentioned embodiments, but it will be understood that the invention can be modified in various way without departing from the sprit and scope of the invention. For example, the ratio determination device 618 can determine whether the ratio of error frames is less than or equal to the predetermined ratio or not by calculating the ratio of error frames instead of the ratio of non-error frames. In this case, the display image control device 614 can display the ultrasonic image G2 combined of the predetermined alternative elastic image EG′ and the B-mode image BG if the ratio of error frames is less than or equal to the predetermined ratio, and it can display the ultrasonic image G3 only including the B-mode image if the ratio of error frames is more than or equal to the predetermined ration.

Further in respective embodiments, regarding non-error frames, the elastic image EG only based on its frame data is displayed, but the elastic image based on data obtained by addition with weighting the color elastic image data of non-error frame which is a current frame and the color elastic image data of the elastic image displayed in the previous frame of the current frame can be displayed. In this case, the weighting coefficient is set larger than of the error frame where the weighting addition is executed for the error frame.

The weighting addition can be executed for the physical amount data before it is scan-converted to the color image elastic image data instead of for the color elastic image data.

The physical amount data processing device 5 can calculate displacement caused by deformation of living tissue or elastic modulus instead of strain as a physical amount related to the elasticity of living tissue, and also calculate the physical amount related to the elasticity of living tissue by other known methods.

Further, the ratio determination device 618 can calculate non-error frames or error frames in multiple frames that does not include the current frame Fn and includes most recent frames. That is, most recent frames backs from the current frame Fn by predetermined number of frames.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a physical amount calculation device configured to calculate a physical amount on elasticity of living tissue based on an echo signal obtained by transmitting an ultrasonic wave to the living tissue; and
a display image control device configured to control display of of a predetermined alternative elastic image displayed instead of an elastic image based on the physical amount calculated for error frame using a ratio of non-error frames or the error frames in a predetermined plurality of frames, wherein the error frames are determined as not meeting a standard.

2. The ultrasonic diagnostic apparatus of claim 1, wherein the predetermined plurality of frames comprises most recent frames including a current frame.

3. The ultrasonic diagnostic apparatus of claim 1, wherein the predetermined plurality of frames comprises most recent frames not including a current frame.

4. The ultrasonic diagnostic apparatus according to claim 1, wherein the predetermined alternative elastic image comprises an image obtained by adding an elastic image of a plurality of frames.

5. The ultrasonic diagnostic apparatus according to claim 2, wherein the predetermined alternative elastic image comprises an image obtained by adding an elastic image of a plurality of frames.

6. The ultrasonic diagnostic apparatus according to claim 3, wherein the predetermined alternative elastic image comprises an image obtained by adding an elastic image of a plurality of frames.

7. The ultrasonic diagnostic apparatus according to claim 1, further comprising a determination device configured to determine whether a frame is an error frame based on whether the elastic image accurately reflects the elasticity of the living tissue.

8. The ultrasonic diagnostic apparatus according to claim 2, further comprising a determination device configured to determine whether a frame is an error frame based on whether the elastic image accurately reflects the elasticity of the living tissue accurately.

9. The ultrasonic diagnostic apparatus according to claim 3, further comprising a determination device configured to determine whether a frame is an error frame based on whether the elastic image accurately reflects the elasticity of the living tissue accurately.

10. The ultrasonic diagnostic apparatus according to claim 4, further comprising a determination device configured to determine whether a frame is an error frame based on whether the elastic image accurately reflects the elasticity of the living tissue.

11. The ultrasonic diagnostic apparatus of claim 5, wherein the physical amount calculation device is configured to set correlation windows on echo signals on the same acoustic ray but being temporally different and to calculate the physical amount by executing a correlation calculation between the correlation windows, the ultrasonic diagnostic apparatus further comprising:
a physical amount average device configured to calculate an average of the physical amount for respective frames; and
a comparison device configured to compare a calculated value obtained from the physical amount average device and a predetermined value of the physical amount, wherein the determination device is further configured to execute the determination based on a result of the comparison device.

12. The ultrasonic diagnostic apparatus of claim 5, wherein the physical amount calculation device is configured to set correlation windows on echo signals on the same acoustic ray but being temporally different and to calculate the physical amount by executing a correlation calculation between the correlation windows, the ultrasonic diagnostic apparatus further comprising:
a correlation coefficient average device configured to calculate an average of a correlation coefficient in an correlation calculation between the correlation windows for respective frames, wherein the determination device executes is configured to execute the determination based on the average value obtained by the correlation coefficient average device.

13. The ultrasonic diagnostic apparatus of claim 5, wherein the physical amount calculation device is configured to set correlation windows on echo signals on the same acoustic ray but being temporally different and to calculate the physical amount by executing a correlation calculation between the correlation windows, the ultrasonic diagnostic apparatus further comprising:
a physical amount average device configured to calculate, by frame, an average of the physical amount obtained for correlation windows in which a correlation calculation of a correlation coefficient more than or equal to a predetermined threshold is executed;
a ratio calculation device configured to calculate a ratio of a calculated value by the physical amount average device to a predetermined average amount of the physical amount;
a correlation coefficient average device configured to calculate an average of the correlation coefficient in an correlation calculation between the correlation windows for respective frames; and
a multiplication device configured to multiply a calculated value of the ratio calculation device and a calculated value of the correlation coefficient average device, wherein the determination device is configured to execute the determination based on a calculated value by the multiplication device.

14. The ultrasonic diagnostic apparatus of claim 5, wherein the physical amount calculation device is configured to set correlation windows on echo signals on the same acoustic ray but being temporally different and to calculate the physical amount using positive and negative signs as the physical amount by executing a correlation calculation between the correlation windows, wherein the determination device is configured to execute the determination based on a ratio of the positive and negative signs in one frame.

15. The ultrasonic diagnostic apparatus of claim 5, wherein the determination device is configured to determine whether a pixel is an error pixel of respective pixels and to determine whether a frame is an error frame based on a ratio of an error pixel or a non-error pixel in one frame.

16. The ultrasonic diagnostic apparatus of claim 15, wherein the determination device is configured to determine whether the is the error pixel based on the physical amount calculated for the respective pixels.

17. The ultrasonic diagnostic apparatus of claim 15, wherein the physical amount calculation device is configured to set correlation windows on echo signals on the same acoustic ray but being temporally different and to calculate the physical amount on respective pixels by executing a correlation calculation between the correlation windows, wherein the determination device is configured to determine whether a pixel of the respective pixels is an error pixel or based on a correlation coefficient in the correlation calculation.

18. The ultrasonic diagnostic apparatus according to claim 1, wherein the display image control device is configured to display, for a non-error frame, the elastic image based on the physical amount calculated from the non-error frame.

19. A method for controlling display of an image comprising:
calculating a physical amount on elasticity of living tissue based on an echo signal obtained by transmitting an ultrasonic wave to the living tissue; and
controlling display of a predetermined alternative elastic image instead of an elastic image based on the physical amount calculated for error frames using a ratio of non-error frames or the error frames in a predetermined plurality of frames, wherein the error frames are determined as not meeting a standard.

20. A computer-readable medium comprising a control program of an ultrasonic diagnostic apparatus configured to instruct a computer to:
calculate a physical amount on elasticity of living tissue based on an echo signal obtained by transmitting an ultrasonic wave to the living tissue; and
control display of a predetermined alternative elastic image instead of an elastic image based on the physical amount calculated for an applicable error frame based a ratio of non-error frames or error frames in a predetermined plurality of frames, wherein error frames are determined as not meeting a standard.
